(19) United States
(12) Patent Application Publication
(10) Pub. No.: US 2026/0096907 A1
(43) Pub. Date: Apr. 9, 2026

(54) Trapezium Implant for Hemiarthroplasty of the Trapeziometacarpal Joint

(71) Applicant: Mark Joseph Warburton, High Point, NC (US)

(72) Inventor: Mark Joseph Warburton, High Point, NC (US)

(21) Appl. No.: 18/906,515

(22) Filed: Oct. 4, 2024

(51) Int. Cl. A61F 2/46; A61B 17/15; A61B 17/17; A61F 2/30; A61F 2/42

(52) U.S. Cl. CPC A61F 2/4606; A61B 17/15; A61B 17/1782; A61F 2002/30116; A61F 2002/30884; A61F 2002/4258

(57) ABSTRACT

A trapezium implant for replacing the distal articular surface of the trapezium at the carpo-metacarpal joint of the thumb by press fit insertion into a prepared channel in a target trapezium, and an insertion guide system and method for inserting the implant. The implant has a saddle-shaped articular surface configured to articulate with a natural existing base of a first metacarpal, articular surface features mimicking natural trapezium bone anatomy and preserving two centers of rotation of the carpo-metacarpal joint, and a single keel extending from a mounting surface and configured to embed the implant into the thickest area of the target trapezium. The single keel minimizes damage to the trapezium and improves surgical implantation. The target trapezium is prepared using an implant insertion guide system including at least one cutting guide and a slot guide, each of which is releasably securable and configured to receive a cutting device.

### FIELD OF THE INVENTION

This invention relates to an implant for replacing the distal articular surface of the trapezium at the carpo-metacarpal joint of the thumb.

### BACKGROUND OF THE INVENTION

Basilar thumb arthritis (arthritis at the base of the thumb) of the carpo-metacarpal (CMC) joint is thought to be the most common arthritis of the homosapien. It typically presents as a symptomatic problem in the sixth decade of life and its incidence increases thereafter. It is a result of the rather recent phylogenetic appearance of a highly mobile strong thumb ray. The thumb acts as the pivotal and guiding member of the defining anatomical features of the human, the prehensile hand. The brain's cortical representation area of the thumb is huge. The thumb allows for a panoply of activities from watch making to weightlifting. Unfortunately, this distinct anatomical advantage can result in arthritis of the CMC joint of the thumb.

In addition to traditional daily activities of the thumb, it is believed that gaming and cell phone text-messaging and the like may also lead to basilar thumb arthritis, with a resulting increase in incidence as well as a potential earlier onset than has been conventional.

Anatomically, the CMC thumb joint is a saddle joint that includes the trapezium articulating with the base of the first metacarpal allowing abduction toward the palm, abduction away from the palm, opposition (toward the 5th finger), and extension or retroposition (backward or hitch-hiker position.) The articular surface of the base of the first metacarpal is divided into dorsal and palmer slopes and a central saddle portion. The opposing articular surface of the trapezium also has two parts: a spherical portion which articulates with the slopes of the first metacarpal, and a saddle portion which articulates with the saddle portion of the first metacarpal. (See, e.g., Zancolli et al., Biomechanics of the trapezio-metacarpal joint, Clinical Orthopaedics and Related Research, No. 220, July 1987, pp. 14-26).

The subchondral (below cartilage) bone of the trapezium-first metacarpal is covered by hyaline cartilage. This cartilage is typically the first tissue to deteriorate during arthritic wear of the joint. Initially, thinning and pitting occurs, which can be followed by osteophyte (bone spur) formation and subluxation (loss of congruity) of the joint.

Over the past fifty years various arthroplasties have been proposed to try to alleviate the disabling pain of CMC arthritis. Generally stated, the arthroplasties have been either soft tissue interpositions, implant interpositions, or partial joint replacements using implants. The implant procedures either have replaced the base of the first metacarpal or replaced the trapezium following trapezectomy.

Currently, first metacarpal implants involve inserting an intramedullary stem into the base of the first metacarpal, to which is attached a convex articular surface replacement. Trapezial implants generally have the shape of the anatomic trapezium. In both cases, an implant material (e.g., metal, silicone, or ceramic) articulates with a bone surface where motion occurs. These procedures do not attempt to replace the joint but rather act as spacers. Fortunately, they can reduce the arthritic pain, but problems have arisen. Potential problems include implant loosening, implant breakage, implant dislocation, adverse tissue reaction to the implant (particularly silicone), failure of pain relief, loss of strength, and implant subsidence (sinking in or erosion of the residual trapezium, as in first metacarpal implants). It is believed that because of these problems, the most common procedure currently performed for CMC arthritis is a soft tissue interposition suspension procedure also known as “ligament reconstruction with tendon interposition (LRTI)” where no implant is used.

### SUMMARY OF THE INVENTION

A trapezium implant for hemiarthroplasty of a carpo-metacarpal (CMC) thumb joint having a resurfacing body with a saddle-shaped articular surface configured to articulate with a natural existing base of a first metacarpal, articular surface features mimicking natural trapezium bone anatomy and preserving two centers of rotation of the carpo-metacarpal joint, a mounting surface engaging a prepared receiving surface of the target trapezium, and a single keel extending from the mounting surface and configured to embed the implant into the thickest area of the target trapezium. The single keel is configured to minimally damage the trapezium and improve surgical implantation procedure by having a length of about 3.85 mm with the length of the stem segment of less than 2.54 mm (0.1 in.). The resurfacing body of the implant has a height at the body center configured to minimize overstuffing of the carpo-metacarpal joint. The trapezium is configured for press fit insertion into a prepared keel channel in the target trapezium.

The target trapezium is prepared using an implant insertion system including at least one cutting guide and a slot guide. The cutting guide is releasably securable within the CMC thumb joint and configured to receive a cutting device to remove of the distal articular surface of the target trapezium to create a flat receiving surface. The cutting guide has a joint guide paddle perpendicularly connected to a cutting guide plate, with the joint guide paddle having at least two sizes configured to fit in the carpo-metacarpal thumb joint. The cutting guide plate abuts at least the radial border of the target trapezium and has a cutting slot positioned a distance below the distal articular surface of the target trapezium along the radial border when the cutting guide is secured within to the CMC thumb joint.

The slot guide is releasably attachable to the target trapezium and configured to receive a cutting device to form the keel channel having a stem channel continuous with an anchor channel in the target trapezium for receiving the stem segment and the anchor segment of the single keel. The slot guide is substantially L-shaped with a first arm configured to run parallel to and engage the flat receiving surface of the target trapezium and a second arm extending downward perpendicularly from the first arm and abutting the radial surface of the target trapezium. A stem slot passes through the second arm at a distance below the first arm and extends continuously upward through end of the second arm and across the first arm to define a slot extending through the first arm and running substantially parallel to the flat receiving surface. An anchor slot passes through the second arm below the stem slot, merging with the stem slot. A stop feature positioned within the second arm of the slot guide protects the target trapezium by limiting ingress of the at least one cutting device.

To replace a distal articular surface of a target trapezium the trapezium implant is inserted by exposing the target trapezium, inserting the cutting guide into the CMC joint of the thumb, removing the distal articular surface of the target trapezium by inserting a surface cutting device into the cutting slot, cutting through the target trapezium creating a cut bone wafer, removing the cutting guide from the CMC thumb joint along with the cut bone wafer thereby leaving a receiving surface on the target trapezium, ensuring the receiving surface is sufficiently flat, securely positioning the slot guide on the receiving surface of the target trapezium, forming a stem channel in the target trapezium using a stem channel cutting device inserted into the stem slot with the stem channel extending from the radial border of the target trapezium to a distance at least half the radial/ulnar length of the target trapezium, forming an anchor channel in the target trapezium using an anchor channel cutting device inserted into the anchor slot with the anchor channel extending from the radial border of the target trapezium to a distance at least half the radial/ulnar length of the target trapezium, verifying dimensions of the receiving surface stem channel and anchor channel, and inserting the trapezium implant into the target trapezium by press fitting the implant into the stem channel and anchor channel to a position recessed about 1.0-2.0 mm from the radial border of the target trapezium.

### BRIEF DESCRIPTION OF THE DRAWINGS

FIG. 1 illustrates a volar view of the skeletal structure of a hand showing the trapezium;

FIG. 2 illustrates a perspective view of the distal articular surface of the trapezium of a left-hand showing radial/ulnar and dorsal/volar orientation;

FIG. 3 illustrates a side view of the trapezium implant showing the saddle shape and single keel in accordance with the invention;

FIG. 4 is a cross-sectional view of the trapezium implant of the present invention;

FIG. 5 is a perspective view of the trapezium implant in accordance with the present invention prior to insertion;

FIG. 6 illustrates a perspective view of the inserted trapezium implant in contact with the articulating base of the first metacarpal bone in accordance with the invention;

FIG. 7 is a perspective view of the cutting guide;

FIG. 8 illustrates the cutting guide inserted between the trapezium and first metacarpal.

FIG. 9 is a perspective view of the slot guide;

FIG. 10 illustrates the slot guide in place upon the cut surface of the trapezium;

FIG. 11 is a perspective view showing the trapezium bone with a cut stem and anchor channel ready for implant insertion;

FIG. 12 illustrates the radius of curvature of the implant;

FIG. 13a is a keel-side view of the implant of the present invention;

FIG. 13b is a perspective view of the implant of the present invention;

FIG. 14a illustrates the CMC joint of the thumb center of abduction-adduction rotation;

FIG. 14b illustrates the CMC joint of the thumb center of flexion-extension rotation;

FIG. 15a is a keel side vie of the implant of the present invention showing a modified mounting surface;

FIG. 15b is a side view of the trapezium implant showing the saddle shape and fenestrated single keel in accordance with the invention; and

FIG. 16 illustrates thumb movements.

### DESCRIPTION OF THE INVENTION

### Parts List

10 trapezium
12 distal articular surface of trapezium
14 radial border of trapezium
20 first metacarpal
30 CMC joint
40 abduction/adduction center of motion
50 flexion/extension center of motion
60 blade/cutting device
100 trapezium implant
102 radial-ulnar length of implant
104 dorsal-volar length of implant
110 resurfacing body
111 body periphery
112 articular surface
114 mounting surface
115 body height
116 surface modifications
120 keel
122 stem segment
123 holes
124 anchor segment
126 leading edge
127 leading edge angle
128 trailing edge
130 trapezium receiving surface
131 keel channel
132 stem channel
134 anchor channel
136 implant insertion guide system
140 cutting guide
141 cutting slot
142 slot guide
143 stem slot
144 anchor slot
145 slot guide stop feature
146 joint guide paddle
147 cutting guide plate
148 first arm
149 second arm
150 stabilizing pins/securing mechanism
151 stabilizing mechanism slots

The disclosed trapezium implant is directed to hemiarthroplasty of the trapeziometacarpal joint (TMC), also known as the CMC joint of the thumb, involving resurfacing of the distal articular surface of the trapezium only. Hemiarthroplasty of the TMC joint preserves the natural saddle shape of the distal articular surface of the trapezium, preserves extensive range of motion of the joint allowed by the saddle shape, preserves the two centers of CMC rotation located at the base of the first metacarpal and base of the trapezium, and eliminates the primary location of osteoarthritis in CMC joint which is at the distal articular surface of the trapezium.

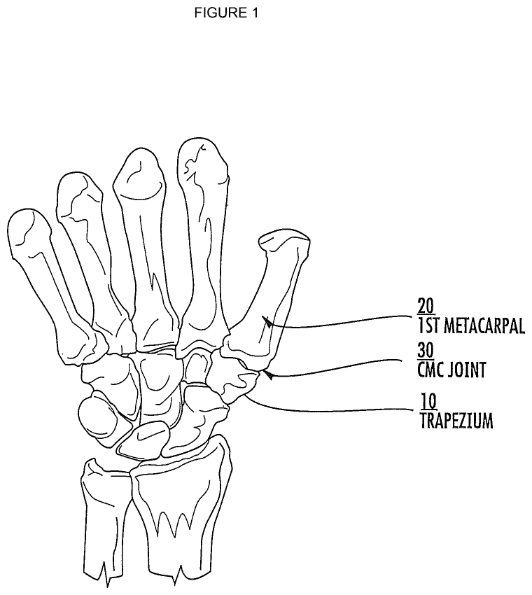

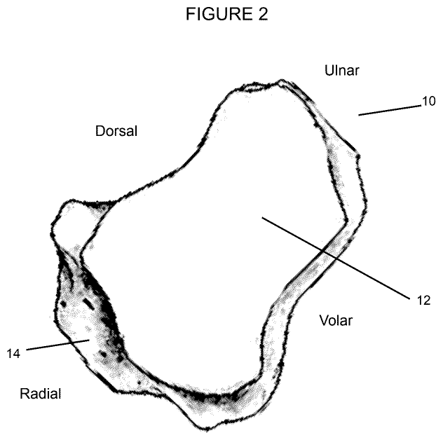

As shown in FIG. 1, the saddle joint (CMC joint 30) is formed by the distal surface of the trapezium bone 10 of the wrist and the base of the first metacarpal bone 20 of the thumb. FIG. 2 illustrates the distal articular surface 12 of the trapezium 10 showing radial/ulnar and dorsal/volar orientation. The disclosed trapezium implant is a press fit, bone ingrowth, articular replacement implant uniquely surfaced to mimic a natural trapezium surface and articulate with the existing, natural base of a first metacarpal bone and is specially configured and inserted with a single keel design to be minimally invasive to the trapezium bone.

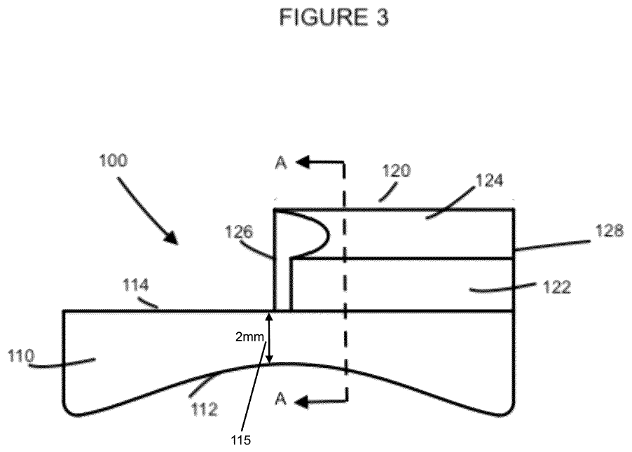

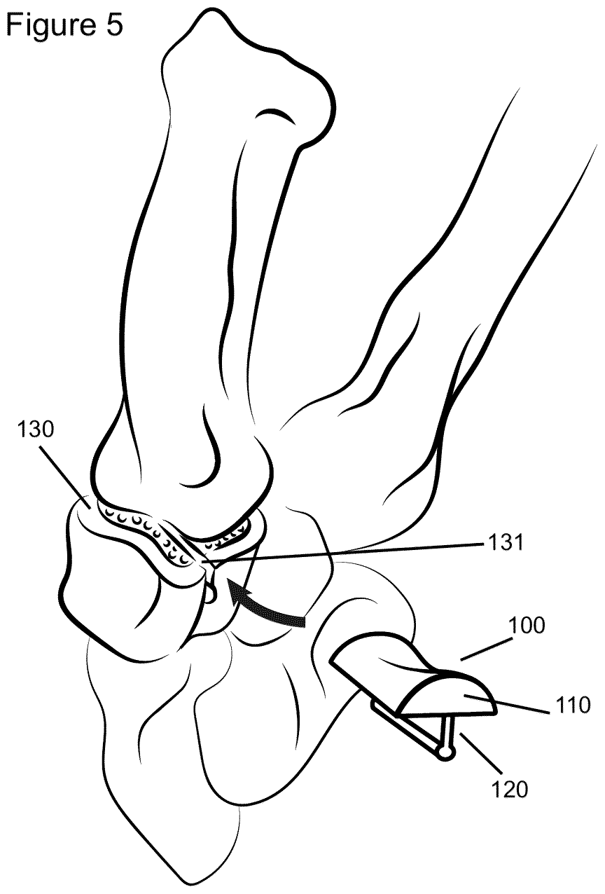

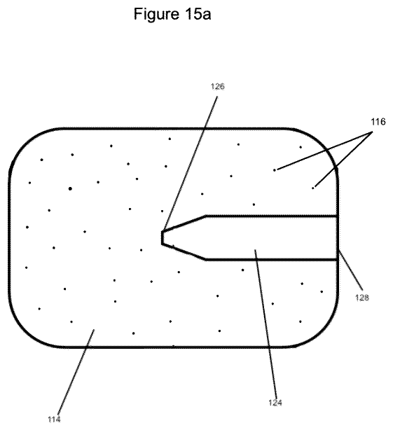

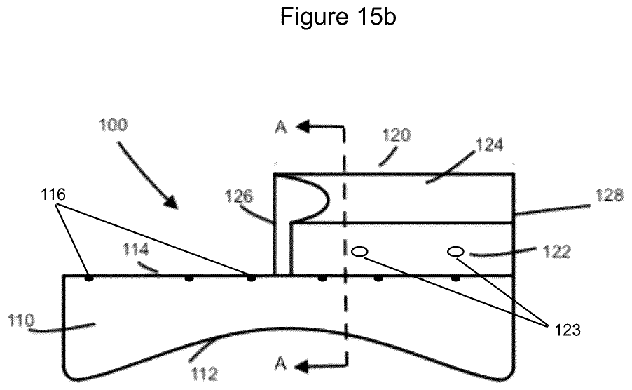

FIG. 3 illustrates a side view of the implant 100 showing the resurfacing body 110 and the keel 120. The resurfacing body 110 comprises an articulating surface 112 and mounting surface 114. Articulating surface 112 is configured to articulate with the base of the first metacarpal while the mounting surface 114 is configured to slidably mount snugly to a prepared trapezium receiving surface 130 as shown in FIG. 5. The keel 120 is comprised of a stem segment 122 and an anchor segment 124 which embeds the implant 100 into the trapezium bone. Keel 120 extends substantially from the central portion of the mounting surface 114 at stem segment 122 and ends at anchor segment 124. In order to promote bone ingrowth, keel 120 and mounting surface 114 can be sintered, roughened, pitted, contoured, or ridged to have surface modifications 116 as shown in FIGS. 15a and 15b. Further, the implant can be modified through treatment with additives such as chemicals or metal fragments. Additionally stem segment 122 can be fenestrated with holes 123 to promote soft tissue and bone ingrowth anchoring as shown in FIG. 15b.

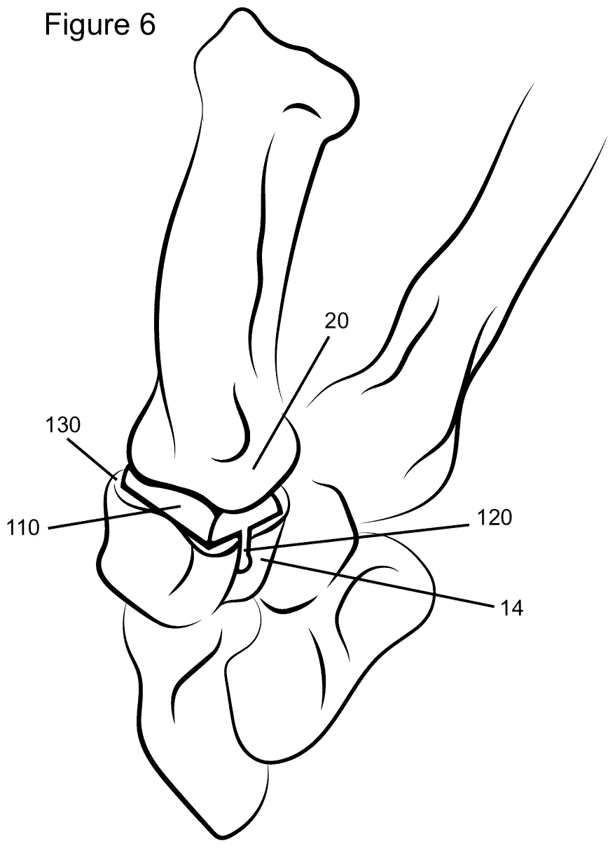

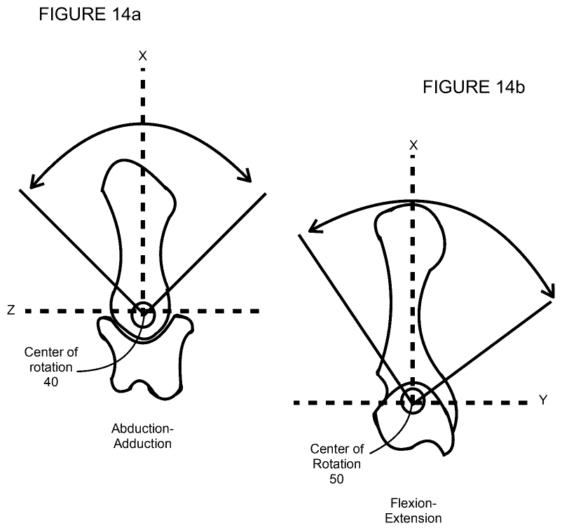

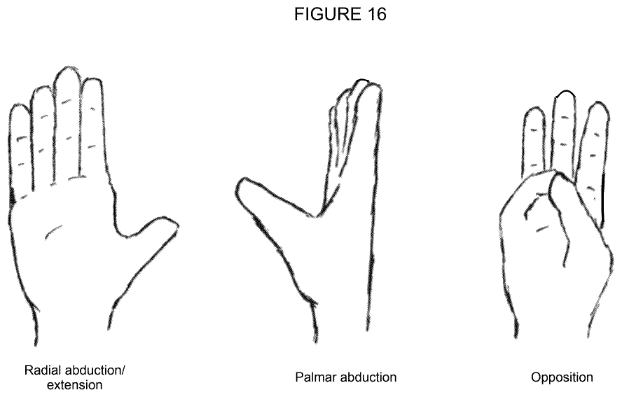

In contrast to known CMC thumb joint implants, the articular surface 112 of implant 100 is designed to complement the natural base of the first metacarpal to create an articulating joint as shown in FIG. 6. Since articular surface 112 articulates with the base of the first metacarpal, articular surface 112 must be polished to ensure optimum functionality and lifespan of the implant. Different types of polishing technologies known in the art can be used to polish articular surface 112. As the trapezium implant 100 of the present invention is not designed to complement a matching man-made metacarpal implant, significant study of anatomical specimens was performed to determine optimal design, shape, and size of the trapezium implant 100 to substantially match the anatomic base of a natural trapezium surface. Trapezium implant 100 must be generic enough to match the various saddle shapes existent in natural trapezium bones yet still distinct enough to articulate with the natural, anatomic base of the first metacarpal. Implant 100 replaces the natural distal articular surface of the trapezium with an anatomical design so as to preserve the two centers of rotation of the CMC joint of the thumb which is necessary for the CMC joint to have a global range of motion allowing for the prehensile thumb ray. As illustrated in FIGS. 14a and 14b, the center of motion for abduction/adduction 40 and the center of motion for flexion/extension 50 are located in different areas. The flexion/extension center 50 is intratrapezial while abduction/adduction center 40 is in the base of the first metacarpal. There is a longitudinal rotation about the longitudinal axis which moves at the same time so that the first metacarpal rotates. As the thumb moves from radial abduction (extension) to palmar abduction and then to opposition as illustrated in FIG. 16, the contact area between the articular surface of the trapezium and the articular surface of the first metacarpal changes.

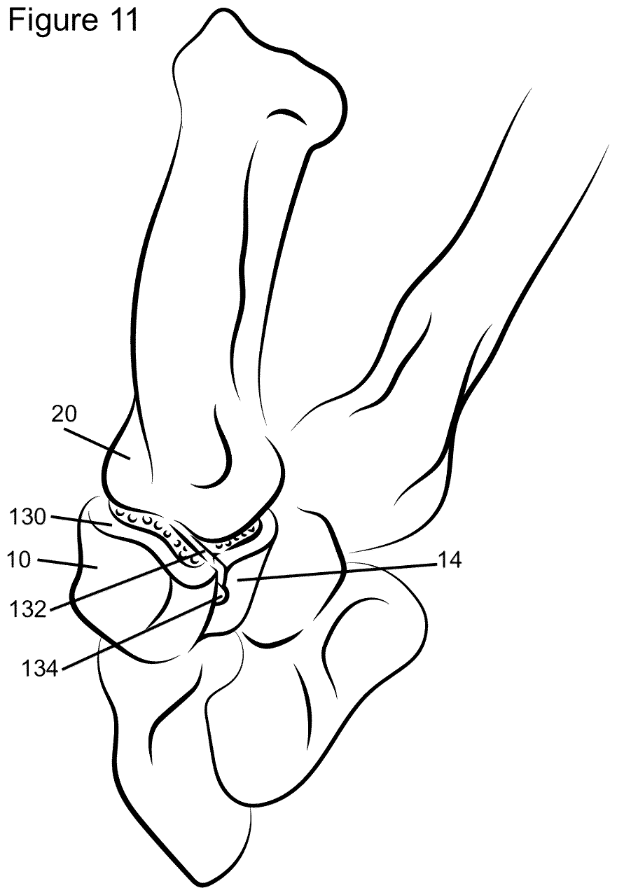

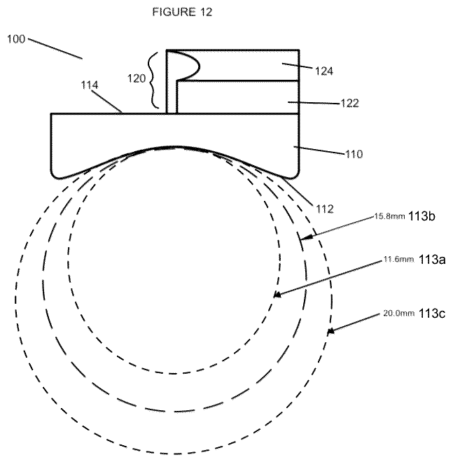

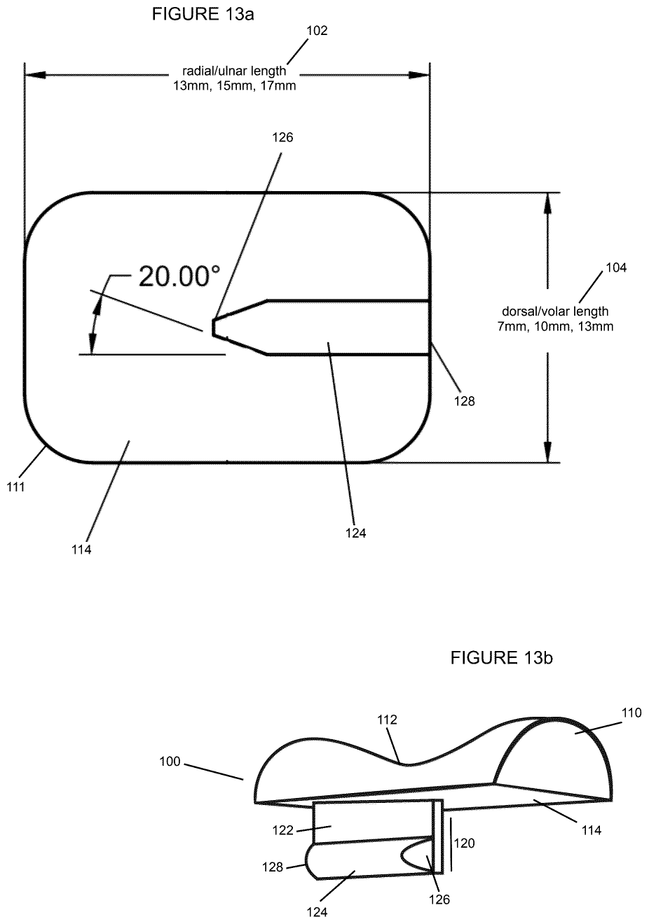

As shown in FIG. 3, articular surface 112 has a general saddle shape but specific anatomic features that mimic the various shapes of natural trapezium bones, critically the radius of curvature. Three distinct radii have been determined to sufficiently encompass the radii of natural trapezium bones. As illustrated in FIG. 12, articular saddle surface 112 demonstrates these distinct radii via three representative circular configurations 113a, 113b, and 113c having preferred diameters of 11.6 mm, 15.8 mm, and 20.0 mm. Further, significant study of anatomical specimens has led to the determination of three sizes for implant 100 based on trapezium radial/ulnar dimensions ranging from 16-29 mm and dorsal/volar dimensions ranging from 12-20 mm of the specimen study group. Based on these studies, the preferred radial/ulnar length 102 of the implant 100 are 13 mm, 15 mm, and 17 mm while the preferred dorsal/volar length 104 of the implant 100 are 7 mm, 10 mm, and 13 mm as illustrated in FIG. 13. Accordingly, implant 100 is preferably provided in sizes having radial/ulnar×dorsal/volar dimensions of 13 mm×7 mm, 15 mm×10 mm, and 17 mm×13 mm. Other suitable implant sizes are possible to provide proper coverage as needed, and may be determined by those skilled in the art. While the established radii of curvature and implant sizes are sufficient for a great majority of implant procedures, custom implants can be provided if needed. Special or unexpected factors such as but not limited to stability and anatomical features may present during the implant procedure that will necessitate a different radius of curvature or implant size. For instance, the particular anatomy of the surface of the first metacarpal could require a customized radius of curvature or implant size that may be determined by those skilled in the art as needed.

Resurfacing body 110 is designed to minimize the height 115 thus reducing the chance of overstuffing the CMC joint. Resurfacing body 110 of implant 100 is preferably about 2 mm in height at the center of the implant as illustrated in FIG. 3. This preferred 2 mm height is consistent regardless of the size of implant 100. Implant 100 is preferably configured so that resurfacing body 110 and keel 120 seamlessly combine to form a solid implant 100. Implant 100 can be manufactured out of various substantially rigid biocompatible materials such as metal, plastic, rigidized polymers, ceramics, silicone, and/or carbon. The preferred implant material is manufactured cobalt chrome; however, other implant material such as but not limited to titanium, stainless steel, and pyrocarbon.

Implant 100 is anchored into the trapezium by a single keel 120. The single keel design requires a less invasive surgery requiring less bone removal which promotes less disruption of trapezium blood supply, shorter surgery time, and a shortened recovery time. The single keel also lessens the chance of an accidental bone fracture which, if undetected, results in post-surgery complications. Further, from a mechanical standpoint, the single keel simplifies the cutting guide design for implant insertion.

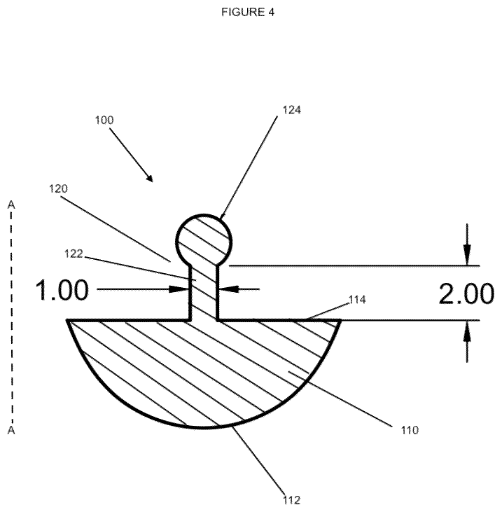

As shown in FIG. 4, keel stem segment 122 is preferably about 2 mm in length and about 1 mm in thickness which minimizes surgical bone damage to the trapezium. As can be seen in FIGS. 3, 13a, and 13b keel 120 extends at least halfway across mounting surface 114 of implant 100, approximately 7 mm-9 mm from the radial border of the trapezium when inserted. As illustrated in FIG. 13a, leading edge 126 of keel 120 is preferably positioned near the center of implant 100 with the trailing edge 128 being positioned in proximity to the edge of implant 100. This positioning of the keel 120 allows the stem segment 122 and anchor segment 124 to reside in the area of the trapezium having thickest bone, namely radial side, while the resurfacing body covers the entirety of the prepared trapezium surface 130. Leading edge 126 of keel 120 is tapered to have a leading edge angle 127 of approximately 20 degrees to facilitate press fit insertion (see FIG. 13a).

To accomplish the positioning of implant 100 in the thickest area of the trapezium, insertion of implant 100 must originate at the radial border of the trapezium. Depending on the size of the trapezium and thus the size of the implant being inserted, keel 120 of implant 100 is inserted into the trapezium to a depth of 7-9 mm from the radial border.

Regardless of the size of the implant being utilized, the overall height [length] of keel 120 including the stem segment 122 and anchor segment 124 is about 3.85 mm. This preferred keel length of about 3.85 mm minimizes damage to the trapezium. Further, width of the keel from the leading edge to the trailing end is about half the total width of the implant. This minimal length/height and width is a significant improvement over previous trapezium implants having keel lengths alone of 2.5 mm up to 6 mm and keel widths extending across substantially more than the center of the implant. Previous trapezium implant configurations reside in too much of the trapezium and jeopardize bone integrity.

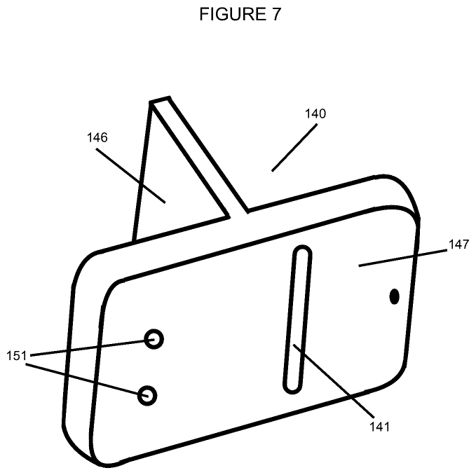

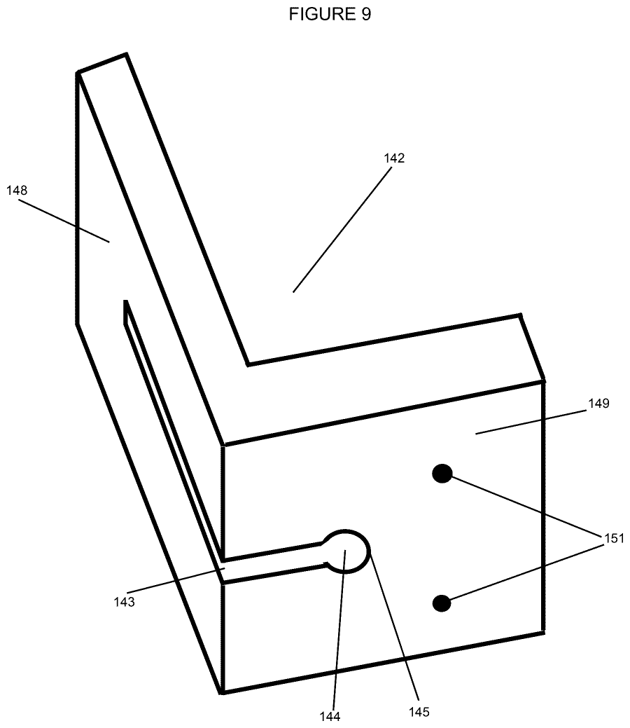

As shown in FIGS. 7 and 9, an implant insertion system 136 comprised of properly sized and specifically configured guides 140 and 142 are needed for surgical accuracy when inserting implant 100 into the trapezium. The cutting guide 140 illustrated in FIG. 7 preferably comes in at least two sizes, each with different joint guide paddle lengths [18, 20 mm]. Both cutting guides require an articular cut of about 4-5 mm. Smaller trapeziums require a smaller articular cut while larger trapeziums require a larger articular cut. Having an option of two cutting guides with different joint guide paddle lengths to engage the articular surface of the trapezium accommodates different trapezium sizes. These guides ensure accurate trapezium articular cuts.

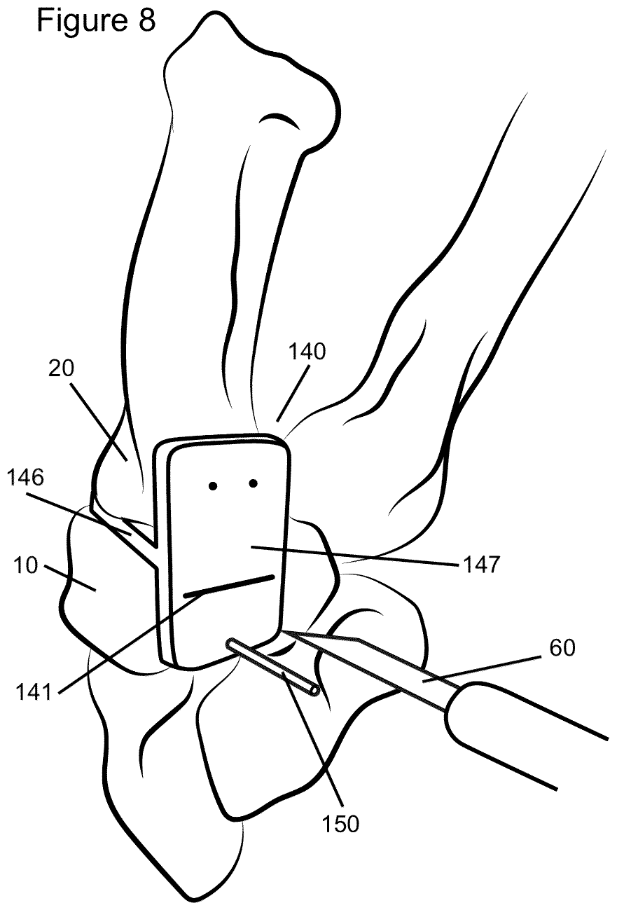

As shown in FIG. 8, the cutting guide 140 is releasably securable within the CMC thumb joint and configured to receive a cutting device 60 to remove of the distal articular surface of the target trapezium to create a flat receiving surface. The cutting guide 140 has a joint guide paddle 146 perpendicularly connected to a cutting guide plate 147. The joint guide paddle 146 fits in the carpo-metacarpal thumb joint while the cutting guide plate 147 abuts the radial side of the trapezium as well as the first metacarpal bone. A cutting slot 141 within the cutting guide plate 147 is positioned a distance of 4-5 mm below the distal articular surface of the target trapezium along the radial border when the cutting guide is secured within to the CMC thumb joint;

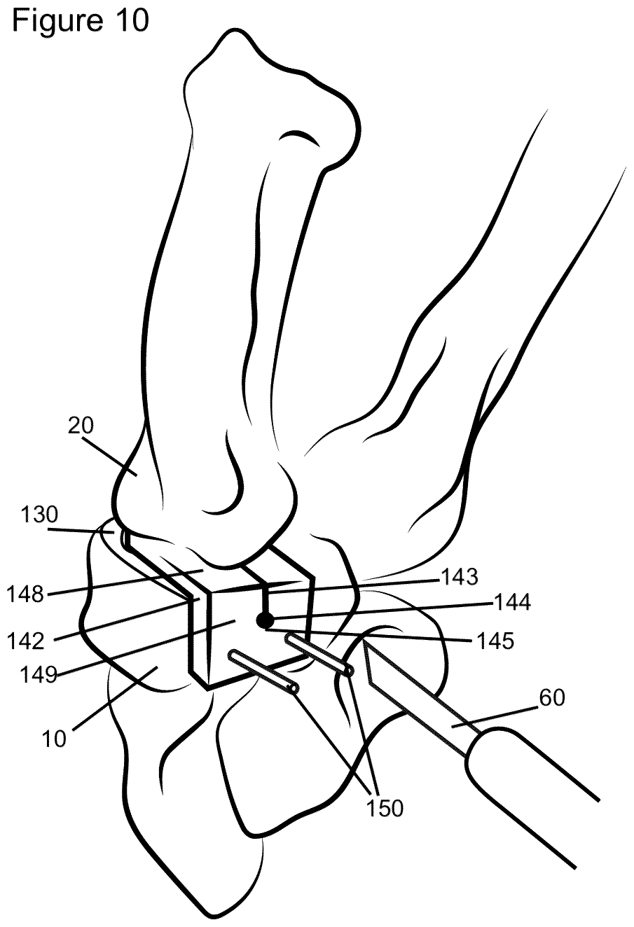

The slot guide 142 has only one slot as shown in FIG. 9 comprising a stem slot 143 for cutting a stem channel 132 into the flat receiving area of the trapezium and an anchor slot 144 for forming anchor channel 134 below the stem channel. The stem channel and anchor channel are cut to match the size, depth, length, and position of the single keel 120 of implant 100. As shown in FIG. 10 the slot guide 142 is releasably attachable to the target trapezium and facilitates the formation of keel channel 131 which is defined by the stem channel 132 continuous with the anchor channel 134 in the target trapezium and receives the stem segment and the anchor segment of the single keel as shown in FIG. 11. The slot guide 142 is substantially L-shaped having a first arm 148 running parallel to and engaging the flat receiving surface 130 of the target trapezium and a second arm 149 extending continuously from the first arm in a downward perpendicular direction and abutting the radial surface of the target trapezium. Each of the first arm and the second arm have respective first and second ends.

As illustrated in FIG. 10, stem slot 143 passes completely through the second arm at a position below the first arm and extends continuously upward through the first end of the second arm and across the first arm to define a slot extending through the first arm and running substantially parallel to the flat receiving surface 130, with the slot continuously running from the second arm to the first arm. The length of the stem slot 143 from the first end of the second arm to the stopping point is approximately equivalent to the length of the keel plus about 2 mm. The width of the stem slot 143 from the second end of the first arm to the end point within the first arm is at least half the width of the trapezium surface and approximately equivalent to the width of the keel. The bottom of the stem slot in the second arm merges into an anchor slot 144 having substantially semicircular cross-section. The bottom of the slot defines a stop feature 145 to protect the target trapezium by limiting ingress of the cutting device into the trapezium.

### Implant Insertion Procedure

Prior to surgery, template pre-op x-rays for implant sizing based on radial/ulnar length of the trapezium are performed on the patient.

Implant 100 is inserted into the radial section of the trapezium via the radial border 14. In order to insert the implant, as illustrated in FIG. 11 the trapezium is prepped by cutting away a portion of the bone to create a flat receiving surface 130 followed by cutting a keel channel defined by stem channel 132 and anchor channel 134 for receiving the keel 120. Once the slots are appropriately cut, the implant can be press fit into the trapezium.

The articular resurfacing hemiarthroplasty of the present invention comprises four steps with press fit insertion of the hemiarthroplasty, namely creating a flat receiving surface on the trapezium bone, creating a stem channel for receiving the stem segment of the single keel, creating an anchor channel for receiving the anchor segment of the single keel, and inserting the implant. Bone ingrowth of the implant insures long term stability and function. Bone ingrowth potential of the implant can be enhanced using a number of techniques that promote bone ingrowth and in vivo stabilization. For example, the mounting surface and keel of the implant can be sintered, roughened, pitted, contoured, or ridged with surface modifications 116 as shown in FIGS. 15a and 15b. Further, the implant can be treated with various additives such as but not limited to chemical or metal fragments. Additionally, the stem segment can be fenestrated with holes 123 to promote soft tissue and bone ingrowth anchoring as illustrated in FIG. 15b.

To access the trapezium, a Wagner incision is made in the volar radial aspect of the CMC joint exposing the thenar muscles. The origins and insertions of the abductor policis brevis and opponens policis are reflected off the trapezium and CMC capsule and base of the first metacarpal (MC). The joint capsule is opened transversely from volar to dorsal and stopped dorsally at the abductor tendon. The capsule and periosteum are reflected off the trapezium proximally, as a flap, to expose enough for implant insertion. Any peri-articular osteophytes are removed so that there is a flat surface of the volar trapezium.

After the trapezium is exposed, cutting guide 140 and slot guide 142 (see FIGS. 7 and 9) are used to prepare the trapezium 10 for receiving the implant. To prepare the trapezium bone, cutting guide 140 is inserted into the CMC joint of the thumb and releasably secured to either or both of the first metacarpal base or the trapezium with a stabilizing or attachment mechanism such as pins 150 as shown in FIG. 8. Using the cutting guide 140, the distal articular surface of the trapezium is osteotomized by inserting cutting device such as blade 60 into cutting slot 141 and cutting through the entirety of the articular distal surface of the trapezium. After the cut is made, the cutting guide is removed along with the cut wafer of bone leaving a flat receiving surface 130 as can be seen in FIG. 11. Cutting guide 140 is available in two sizes to accommodate a range of trapezium sizes. Extra-large trapeziums (>22 mm) require an extra-large cutting guide.

Once the trapezium is cut, slot guide 142 and a metal ruler are used to ensure that the cut surface is flat all the way over to the second metacarpal. The receiving surface 130 can be trimmed with a saw blade as needed to achieve a sufficiently flat cut surface. Next, the slot guide 142 is releasably attached to receiving surface 130, as can be seen in FIG. 10. Slot guide 142 is preferably held in place using stabilizing pins 150 though any attachment mechanism known in the art. When slot guide 142 is in place, cutting devices are used to form a keel channel 131 in the trapezium. This keel channel 131 is preferably defined by two separate but merging channels, namely a stem channel 132 and an anchor channel 134. Typically, a saw blade is inserted into stem slot 143 to cut a stem channel 132, and a drill is inserted into anchor slot 144 to configure anchor channel 134 as shown in FIG. 10, but any cutting devices known in the art could be used. The bottom of anchor slot 144 serves as a stop feature 145 to protect the trapezium by limiting the ingress of the saw blade into the trapezium. Stop feature 145 stops the cutting device at a distance of the length of the single keel plus 2 mm, thus ensuring sufficient depth of the channel for receive the keel while minimizing damage to the trapezium. As shown in FIG. 5, stem channel 132 is cut to the appropriate depth to receive the stem segment 122 of keel 120 of the implant 100, and anchor channel 134 is configured to receive anchor segment 124 of keel 120. The slot guide 142 is removed, and a modified trial implant and fluoroscopy is used to verify cut/slot depth and size of the implant. Finally, implant 100 is inserted into the trapezium as shown in FIG. 6 so that it is recessed from the border of the trapezium, preferably about 1-2 mm. Impaction may be necessary to securely fit the implant.

After the implant is secure, the wounds are irrigated, the reflected capsule and periosteum of the trapezium is sutured back to the base of the first metacarpal, and the thenar muscles are sutured back to the radial capsule and the base of the first metacarpal. Wounds are closed with 5-0 nylon, and a plaster volar thumb spica splint is applied.

The use of the terms “a” and “an” and “the” and similar references in the context of this disclosure are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure, and does not pose a limitation on the scope of the claims. No language in the specification should be construed as indicating any non-claimed element as essential to the practice of the preset disclosure.

Multiple embodiments are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The use of individual numerical values are stated as approximations as though the values were preceded by the word “about”, “substantially”, or “approximately.” Similarly, the numerical values in the various ranges specified in this application, unless expressly indicated otherwise, are stated as approximations as though the minimum and maximum values within the stated ranges were both preceded by the word “about”, “substantially”, or “approximately.” In this manner, variations above and below the stated ranges can be used to achieve substantially the same results as values within the ranges. As used herein, the terms “about”, “substantially”, and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about”, “substantially”, or “approximately” will serve to broaden a particular numerical value or range. Thus, as a general matter, “about”, “substantially”, or “approximately” broaden the numerical value. Also, the disclosure of ranges is intended as a continuous range including every value between the minimum and maximum values plus the broadening of the range afforded by the use of the term “about”, “substantially”, or “approximately”. Thus, recitation of ranges of values herein are merely intended to serve as a shorthand method of referring individually to each separate value falling within the range, unless otherwise indicated herein, and each separate value is incorporated into the specification as if it were individually recited herein. To the extent that determining a given amount of variation of some the factors. as well as other considerations known to those of skill in the art to which the disclosed subject matter is most closely related or the art relevant to the range or element at issue will have on the performance of the claimed subject matter, is not considered to be within the ability of one of ordinary skill in the art, or is not explicitly stated in the claims, then the terms “about”, “substantially”; and “approximately” should be understood to mean the numerical value, plus or minus 10%.

It is to be understood that any ranges, ratios, and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented.

What is claimed is:

1. A trapezium implant for hemiarthroplasty of a carpo-metacarpal (CMC) thumb joint comprising:
a. a resurfacing body having:
i. a periphery;
ii. a body center substantially equidistant from said periphery;
iii. a dorsal/volar length corresponding to a dorsal/volar orientation of a target trapezium, said target trapezium having a radial border;
iv. a radial/ulnar length corresponding to a radial/ulnar orientation of the target trapezium;
v. a saddle-shaped articular surface defining a distal articular surface of the target trapezium configured to articulate with a natural existing base of a first metacarpal;
vi. articular surface features mimicking natural trapezium bone anatomy, said articular surface features preserving two centers of rotation of the carpo-metacarpal joint; and
vii. a mounting surface engaging a prepared receiving surface of said target trapezium, said mounting surface having a central portion spanning said dorsal/volar length through the body center; and
b. a single keel extending substantially from said central portion of said mounting surface along the radial/ulnar length and configured to embed said implant into said target trapezium, said single keel having:
i. a leading edge positioned approximately at the body center of the mounting surface of said resurfacing body;
ii. a trailing edge positioned proximate the periphery of said resurfacing body;
iii. a width between said leading edge and said trailing edge being substantially half the radial/ulnar length of the resurfacing body;
iv. a stem segment having a first end proximal said mounting surface, a second end distal said mounting surface, a length between said first end and said second end, and a thickness;
v. an anchor segment extending from said second end of said stem segment;
wherein, said resurfacing body has a height at the body center configured to minimize overstuffing of the carpo-metacarpal joint;
wherein said single keel has a length of about 3.85 mm, said length including the stem segment and the anchor segment;
wherein, said length of said stem segment is less than 2.54 mm (0.1 in.);
wherein, said single keel is configured to minimally damage the trapezium and improve surgical implantation procedure;
wherein, said single keel is positioned with said mounting surface to allow said stem segment and said anchor segment to reside in a thickest area of the target trapezium;
wherein, the resurfacing body is configured to substantially cover the prepared trapezium surface; and
wherein, the leading edge of the single keel facilitates press fit insertion of said implant into a prepared keel channel in the target trapezium, said prepared keel channel extending from the radial border of the target trapezium.

2. The trapezium implant of claim 1 wherein said articular surface features comprise shapes, angles, and dimensions configured to mimic a radius of curvature naturally present in the target trapezium.

3. The trapezium implant of claim 2 wherein said radius of curvature is in the range of about 11.5 mm to about 20.0 mm.

4. The trapezium implant of claim 3 wherein said radius of curvature is chosen from: 11.5 mm, 15.8 mm, and 20.0 mm.

5. The trapezium implant of claim 1 wherein said dorsal/volar length is in the range of about 12 mm to about 20 mm and said radial/ulnar length is in the range of about 16 mm to about 29 mm.

6. The trapezium implant of claim 5 wherein the dorsal/volar length by the radial/ulnar length is chosen from: 7 mm×13 mm, 10 mm×15 mm, and 13 mm×17 mm.

7. The trapezium implant of claim 1 wherein the height at the body center of said resurfacing body is about 2 mm.

8. The trapezium implant of claim 1 wherein said leading edge is tapered at an angle of approximately 20 degrees.

9. The trapezium of claim 1 wherein the width between said leading edge and said trailing edge of said single keel extends about 7 mm to about 9 mm from the radial border of the target trapezium.

10. The trapezium of claim 1 wherein said anchor segment has a substantially semicircular cross section.

11. The trapezium of claim 1 wherein said prepared keel channel is defined by a stem channel that merges into an anchor channel.

12. The trapezium of claim 1 further comprising at least one modification to the implant to promote bone ingrowth, said at least one modification being chosen from: sintering, roughening, pitting, and contouring at least the mounting surface, treating at least the mounting surface with chemical additives and metal fragments, and incorporating a fenestrated stem segment.

13. The trapezium implant of claim 1 wherein said surgical implantation procedure is improved by requiring less bone removal, promoting less disruption of trapezium blood supply, lessening chance accidental bone fracture, shorter surgery time, and shortened recovery time.

14. The trapezium implant of claim 1 further comprising an implant insertion system comprising:
a. at least one cutting guide releasably securable within the CMC thumb joint and configured to receive a cutting device to remove of the distal articular surface of the target trapezium to create a flat receiving surface, said cutting guide having:
i. a joint guide paddle perpendicularly connected to a cutting guide plate, said joint guide paddle configured to fit in the carpo-metacarpal thumb joint;
ii. a cutting slot within said cutting guide plate, said cutting guide plate configured to abut at least the radial border of the target trapezium with said cutting slot positioned a distance below the distal articular surface of the target trapezium along the radial border when the cutting guide is secured within to the CMC thumb joint;
b. a slot guide releasably attachable to the target trapezium and configured to receive at least one cutting device to form said prepared keel channel having a stem channel continuous with an anchor channel in the target trapezium for receiving the stem segment and the anchor segment of the single keel, said slot guide being substantially L-shaped and having:
i. a first arm having a first end and a second end and configured to run parallel to and engage the flat receiving surface of the target trapezium;
ii. a second arm having a first end and a second end, said second arm extending downward perpendicularly from said second end of the first arm and abutting the radial surface of the target trapezium;
iii. a stem slot passing through said second arm at a distance below the first arm and extending continuously upward through the first end of the second arm and across said first arm to define a slot extending through the first arm and running substantially parallel to said flat receiving surface from said second end of the first arm to a point proximal said first end of the first arm;
iv. an anchor slot passing through said second arm below said stem slot, said stem slot merging into said anchor slot;
v. a stop feature positioned within the second arm of the slot guide, said stop feature configured to protect the target trapezium by limiting ingress of the at least one cutting device.

15. The trapezium implant of claim 14 wherein said at least one cutting guide is a cutting guide having one of: a joint guide paddle 18 mm in length and a cutting guide having a joint guide paddle 20 mm in length.

16. The trapezium implant of claim 14 wherein said at least one cutting device to form a stem channel continuous with an anchor channel in the target trapezium comprises a saw blade and a drill.

17. The trapezium implant of claim 14 further comprising at least one stabilizing mechanism configured to temporarily secure said cutting guide and said slot guide in a preferred position.

18. The trapezium implant of claim 14 wherein the distance below the distal articular surface of the target trapezium of said cutting slot is 4-5 mm when the cutting guide is secured within to the CMC thumb joint.

19. A method for hemiarthroplasty of a carpo-metacarpal (CMC) thumb joint that replaces a distal articular surface of a target trapezium with a trapezium implant, the comprising:
a. exposing the target trapezium through an incision made in the volar radial aspect of the CMC joint, said target trapezium having a dorsal/volar length and a radial/ulnar length;
b. inserting a cutting guide into the CMC joint of the thumb, said cutting guide comprising:
i. a joint guide paddle perpendicularly connected to a cutting guide plate, said joint guide paddle configured to fit in the CMC thumb joint; and
ii. a cutting slot within said cutting guide plate, said cutting guide plate configured to abut at least a radial border of the target trapezium with said cutting slot positioned a distance below the distal articular surface of the target trapezium along the radial border when the cutting guide is inserted;
c. removing the distal articular surface of the target trapezium by inserting a surface cutting device into the cutting slot of said cutting guide, cutting through the target trapezium creating a cut bone wafer, and removing the cutting guide from the CMC thumb joint along with the cut bone wafer, wherein removing the distal articular surface leaves a receiving surface on the target trapezium;
d. ensuring the receiving surface is sufficiently flat;
e. securely positioning a slot guide on the receiving surface of the target trapezium, said slot guide being substantially L-shaped and comprising:
i. a first arm having a first end and a second end and configured to run parallel to and engage the receiving surface of the target trapezium;
ii. a second arm having a first end and a second end, said second arm extending downward perpendicularly from said second end of the first arm and abutting the radial surface of the target trapezium;
iii. a stem slot passing through said second arm at a distance below the first arm and extending continuously upward through the first end of the second arm and across said first arm to define a slot extending through the first arm and running substantially parallel to said flat receiving surface from said second end of the first arm to a point near said first end of the first arm;
iv. an anchor slot passing through said second arm below said stem slot, said stem slot merging into said anchor slot;
v. a stop feature positioned within the first arm of the slot guide, said stop feature configured to protect the target trapezium by limiting ingress of the at least one cutting device;
f. forming a stem channel in the target trapezium using a stem channel cutting device inserted into the stem slot of said slot guide, said stem channel extending from the radial border of the target trapezium to a distance at least half the radial/ulnar length of the target trapezium;
g. forming an anchor channel in the target trapezium using an anchor channel cutting device inserted into the anchor slot of said slot guide, said anchor channel extending from the radial border of the target trapezium to a distance at least half the radial/ulnar length of the target trapezium;
h. verifying dimensions of the receiving surface stem channel and anchor channel; and
i. inserting the trapezium implant into the target trapezium by press fitting the implant into the stem channel and anchor channel to a position recessed about 1.0-2.0 mm from the radial border of the target trapezium, said trapezium implant comprising:
i. a resurfacing body having:
A. a periphery;
B. a body center substantially equidistant from said periphery;
C. a dorsal/volar length corresponding to a dorsal/volar orientation of a target trapezium;
D. a radial/ulnar length corresponding to a radial/ulnar orientation of the target trapezium;
E. a saddle-shaped articular surface defining a distal articular surface of the target trapezium configured to articulate with a natural existing base of a first metacarpal;
F. articular surface features mimicking natural trapezium bone anatomy, said articular surface features preserving two centers of rotation of the CMC thumb joint; and
G. a mounting surface engaging the receiving surface of said target trapezium, said mounting surface having a central portion spanning said dorsal/volar length through the body center; and
ii. a single keel extending substantially from said central portion of said mounting surface along the radial/ulnar length and configured to embed said implant the target trapezium, said single keel having:
A. a leading edge positioned approximately at the body center of the mounting surface of said resurfacing body;
B. a trailing edge positioned proximate the periphery of said resurfacing body;
C. a width between said leading edge and said trailing edge being substantially half the radial/ulnar length of the resurfacing body;
D. a stem segment having a first end proximal said mounting surface, a second end distal said mounting surface, a length between said first end and said second end, and a thickness, said stem segment configured to fit snugly within the stem channel;
E. an anchor segment extending from said second end of said stem segment, said anchor segment configured to fit snugly within the anchor channel;
wherein, said resurfacing body has a height at the body center configured to minimize overstuffing of the CMC joint;
wherein said single keel has a length of about 3.85 mm, said length including the stem segment and the anchor segment;
wherein, said length of said stem segment is less than 2.54 mm (0.1 in.);
wherein, said single keel is configured to minimally damage the trapezium and improve surgical implantation procedure;
wherein, said single keel is positioned with said mounting surface to allow said stem segment and said anchor segment to reside in a thickest volar area of the target trapezium; and
wherein, the resurfacing body is configured to substantially cover the prepared trapezium surface.
